(19) United States
(12) Patent Application Publication
(10) Pub. No.: US 2009/0118542 A1
(43) Pub. Date: May 7, 2009

(54) Process for Production of Optically Active Carboxlic Acid Compound

(76) Inventors: Shigeya Yamazaki, Settsu-shi (JP); Takeshi Hosoya, Nishinomiya-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/224,043

(22) PCT Filed: Feb. 15, 2007
(86) PCT No.: PCT/JP2007/052718

§ 371 Date: Sep. 24, 2008

(30) Foreign Application Priority Data
Feb. 16, 2006 (JP) 2006-039867

(51) Int. Cl. C07C 51/00

(52) U.S. Cl. 562/599

(57) ABSTRACT

An optically active acylated camphorsultam can be hydrolyzed with an aqueous solution of an alkaline earth metal hydroxide in the presence of a branched alkanol, to give a corresponding optically active carboxylic acid compound in safely and inexpensively.

### TECHNICAL FIELD

The present invention relates to a process for producing an optically active carboxylic acid compound. More specifically, the present invention relates to a process comprising hydrolyzing an acylated camphorsultam to produce a corresponding optically active carboxylic acid compound.

### BACKGROUND ART

(2R)-2-propyloctanoic acid (generic name: arundic acid) represented by

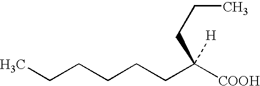

is a compound useful as a medicament, for example, as a therapeutic medicament for brain infarction and amyotrophic lateral sclerosis.

In order to efficiently produce the above-mentioned optically active carboxylic acid, processes in which an acylated camphorsultam is used have been developed. For example, (1) a process for treating an acylated camphorsultam with alkali metal hydroxide in water-miscible solvent in the presence of peroxy acid (aqueous hydrogen peroxide solution) and (2) a process for treating an acylated camphorsultam with tetraalkylammonium hydroxide (for example, tetrabutylammonium hydroxide) in water-miscible organic solvent in the presence of peroxy acid (for example, aqueous hydrogen peroxide solution) are disclosed in WO99/58513.

According to the above-mentioned process (1) or (2), (2S)-2-(2-propenyl)octanoic acid (Example 3), (2S)-2-(2-propynyl)octanoic acid (Examples 8(a), (b)), and (2R)-2-propyloctanoic acid (Example 6) are produced from N-[(2S)-2-(2-propenyl)octanoyl]-(1S)-(−)-2,10-camphorsultam, N-[(2S)-2-(2-propynyl)octanoyl]-(1S)-(−)-2,10-camphorsultam, and N-[(2R)-2-propyloctanoyl]-(1S)-(−)-2,10-camphorsultam, respectively.

However, the above-mentioned process (1) is not an industrially preferable process because in the case the acylated camphorsultam is N-[(2S)-2-(2-propenyl)octanoyl]-(1S)-(−)-2,10-camphorsultam, for example, hydrolysis does not proceed efficiently, the recovery rate of the camphorsultam is low, and the recovered camphorsultam must be reduced with sodium borohydride (SBH) or the like before reuse due to contained camphorsulfonimine, which is an imine form thereof.

In addition, the above-mentioned process (2) has a problem in industrial practice because this process produces oxygen that might cause explosion, and therefore, in a low-flash-point solvent system, oxygen concentration should be reduced to a safe level by running a large amount of nitrogen gas. Further, in order to avoid the risk of explosion by employing a high-flash-point solvent, expensive solvents such as diglyme must be used, which is also a problem in industrial practice.

### DISCLOSURE OF THE INVENTION

### Problems to be Solved by the Invention

An object of the present invention is to provide a novel process wherein an acylated camphorsultam is safely hydrolyzed at low cost to produce a corresponding optically active carboxylic acid compound, and the camphorsultam obtained as a by-product is recovered in high yield.

### Means for Solving the Problems

To achieve the above-mentioned object, the present inventors have studied intensively, and as a result, have found that in a process comprizing hydrolyzing an acylated camphorsultam to produce a corresponding optically active carboxylic acid compound, when an alkaline earth metal hydroxide such as barium hydroxide, calcium hydroxide and strontium hydroxide is used as the base, hydrolysis proceeds efficiently and safely even without peroxy acid such as hydrogen peroxide, in the presence of low-cost branched alkanol such as isopropanol and tertiary butanol, and that high-purity camphorsultam can be recovered at a high recovery rate. The present invention has been completed based on further studies.

That is, the present invention is as follows:

(1) a process for producing an optically active carboxylic acid compound represented by the general formula [II],

R—COOH [II]

wherein R represents an optically active hydrocarbon group having at least one asymmetric carbon atom and optionally having at least one substituent, which comprises hydrolyzing an acylated camphorsultam represented by the general formula [I]

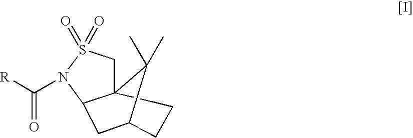

with an aqueous solution of an alkaline earth metal hydroxide in the presence of a branched alkanol, wherein R has the same meaning as described above;
(2) the process according to the above-mentioned (1), wherein the alkaline earth metal hydroxide is barium hydroxide, calcium hydroxide or strontium hydroxide;
(3) the process according to the above-mentioned (1) or (2), wherein the branched alkanol is a secondary alkanol or a tertiary alkanol;
(4) the process according to the above-mentioned (1) or (2), wherein the branched alkanol is isopropanol or tertiary butanol;
(5) the process according to the above-mentioned (1), wherein the alkaline earth metal hydroxide is barium hydroxide or calcium hydroxide, and the branched alkanol is isopropanol;
(6) the process according to any one of the above-mentioned (1) to (5), wherein R is an optically active hydrocarbon group having at least one asymmetric carbon atom and optionally having at least one substituent selected from the group consisting of an alkyl group, an alkoxy group, an optionally protected hydroxy group, an aryl group, an aryloxy group, an aralkyloxy group, an optionally protected amino group, a monoalkylamino group, a dialkylamino group, an acylamino group, an oxo group, an alkylthio group, and an arylthio group;
(7) the process according to any one of the above-mentioned (1) to (6), wherein the hydrocarbon group is a saturated or unsaturated acyclic hydrocarbon group, or a saturated or unsaturated cyclic hydrocarbon group;
(8) the process according to the above-mentioned (7), wherein the saturated or unsaturated acyclic hydrocarbon group is a linear or branched alkyl group, a linear or branched alkenyl group, or a linear or branched alkynyl group;
(9) the process according to any one of the above-mentioned (1) to (5), wherein R is an optically active branched alkenyl group having at least one asymmetric carbon atom and optionally having at least one substituent;
(10) the process according to any one of the above-mentioned (1) to (5), wherein R is a group represented by the formula,

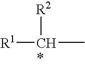

wherein R¹ represents a C1 to C10 alkyl group; R² represents a C1 to C6 alkyl group, a C2 to C6 alkenyl group, or a C2 to C6 alkynyl group; and * represents an asymmetric carbon atom;
(11) the process according to the above-mentioned (10), wherein R² is a C2 to C6 alkenyl group;
(12) the process according to any one of the above-mentioned (1) to (5), wherein the acylated camphorsultam represented by the general formula [I] is an acylated camphorsultam represented by the formula [I-A],

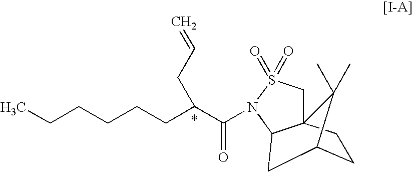

wherein * represents an asymmetric carbon atom,
and the optically active carboxylic acid compound represented by the general formula [II] is an optically active 2-(2-propenyl)octanoic-acid represented by the formula [II-A],

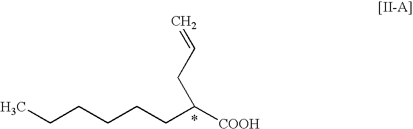

wherein * represents an asymmetric carbon atom;
(13) the process according to the above-mentioned (12), wherein the configurations of the asymmetric carbon atoms bound to the 2-propenyl group in the optically active 2-(2-propenyl)octanoic acid represented by the formula [II-A] and in the acylated camphorsultam represented by the formula [I-A] are S configurations;
(14) the process according to the above-mentioned (12) or (13), wherein the branched alkanol is isopropanol and the alkaline earth metal hydroxide is calcium hydroxide; and
(15) the process according to any one of the above-mentioned (1) to (14), wherein the camphorsultam represented by the formula [III]

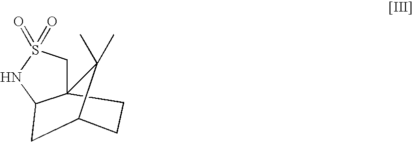

is additionally recovered from the post-reaction solution after hydrolysis.

### BRIEF DESCRIPTION OF THE DRAWINGS

FIG. 1 is an XRD chart of Crystal Form 1 of cyclohexylamine salt of (2S)-2-(2-propenyl) octanoic acid.

FIG. 2 shows an XRD chart of Crystal Form 2 of cyclohexylamine salt of (2S)-2-(2-propenyl) octanoic acid.

### BEST MODE FOR CARRYING OUT THE INVENTION

According to the present invention, an optically active carboxylic acid compound represented by the general formula [II],

R—COOH [II]

wherein R represents an optically active hydrocarbon group having at least one asymmetric carbon atom and optionally having at least one substituent, which comprises hydrolyzing an acylated camphorsultam represented by the general formula [I]

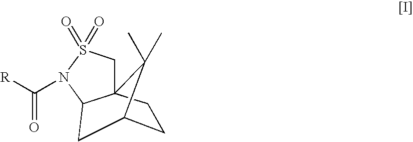

with an aqueous solution of an alkaline earth metal hydroxide in the presence of a branched alkanol, wherein R has the same meaning as described above can be produced.

In the acylated camphorsultam represented by the general formula [I], which is a raw material compound of the present invention, the camphorsultam part may be (1S)-(−)-2,10-camphorsultam or (1R)-(+)-2,10-camphorsultam.

In addition, in the acylated camphorsultam represented by the general formula [I], which is a raw material compound of the present invention, the group represented by R is “an optically active hydrocarbon group having at least one asymmetric carbon atom and optionally having at least one substituent”.

The “hydrocarbon group” in the above-mentioned “an optically active hydrocarbon group having at least one asymmetric carbon atom and optionally having at least one substituent” may include a saturated or unsaturated acyclic hydrocarbon group, and a saturated or unsaturated cyclic hydrocarbon group. Herein, the saturated or unsaturated acyclic hydrocarbon group may include, for example, a linear or branched alkyl group, a linear or branched alkenyl group, and a linear or branched alkynyl group. The total number of carbon atoms in these groups is 20 or less, preferably 16 or less. In particular, for example, a group represented by the formula,

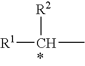

wherein R¹ represents a C1 to C10 alkyl group; R² represents a C1 to C6 alkyl group, a C2 to C6 alkenyl group, or a C2 to C6 alkynyl group; and * represents an asymmetric carbon atom is included, and more particularly, for example, a group represented by the following formula,

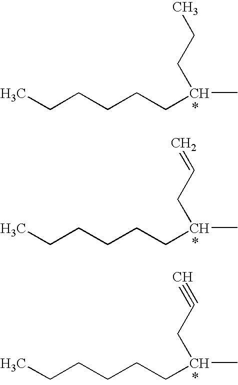

wherein * represents an asymmetric carbon atom is included.

The saturated or unsaturated cyclic hydrocarbon group may include, for example, a cycloalkyl group, a bicycloalkyl group, a cycloalkenyl group, and a bicycloalkenyl group. The total number of carbon atoms in these groups is 20 or less, preferably 12 or less. In particular, for example, a group represented by the following formula,

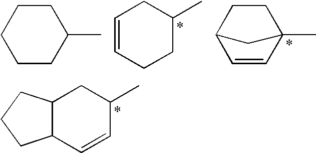

wherein * represents an asymmetric carbon atom is included.

The “substituent” in the above-mentioned “an optically active hydrocarbon group having at least one asymmetric carbon atom and optionally having at least one substituent” may include an alkyl group, an alkoxy group, a hydroxy group, an aryl group, an aryloxy group, an aralkyloxy group, an amino group, a monoalkylamino group, a dialkylamino group, an acylamino group, an oxo group, an alkylthio group, and an arylthio group. Among these, the alkoxy group may include C1 to C6 alkoxy groups; the aryl part of the aryl group, the aryloxy group, and the arylthio group may include, for example, a phenyl group and a naphthyl group; the aralkyl part of the aralkyloxy group may include, for example, a benzyl group and a phenethyl group; the alkyl part of the monoalkylamino group, the dialkylamino group, and the alkylthio group may include, for example, C1 to C6 alkyl groups; and the acyl group of the acylamino group may include, for example, C2 to C6 alkanoyl groups. Also, the hydrocarbon group may have one or more the above-mentioned substituents, which may be the same or different. As a result of substitution with these substituents, the “hydrocarbon group” may become asymmetric.

The “branched alkanol” used as a solvent in the hydrolysis of the present invention includes C3 to C6 secondary alkanols (for example, isopropanol, isobutyl alcohol and isopentylalcohol) and C4 to C6 tertiary alkanols (for example, tertiary butanol and tertiary pentanol). Among these, isopropanol and tertiary butanol are preferable, and isopropanol is most preferable.

Also, the “alkaline earth metal hydroxide” used in the hydrolysis of the present invention includes barium hydroxide, calcium hydroxide and strontium hydroxide. Among these, barium hydroxide and calcium hydroxide are preferable, and calcium hydroxide is the most preferable.

The preferable amount of the alkaline earth metal hydroxide used in the hydrolysis reaction of the present invention is in the range of 0.5 to 5.0 mol, preferably in the range of 1.0 to 3.0 mol, more preferably in the range of 1.0 to 2.5 mol, and especially preferably in the range of 1.0 to 2.0 mol, per 1 mol of the acylated camphorsultam represented by the general formula [I]. Meanwhile, while the used amount of the branched alkanol may be any as long as it is sufficient to dissolve the acylated camphorsultam represented by the general formula [I], it is usually 300 to 1500 parts by volume and preferably 400 to 800 parts by volume per 100 parts by weight of the acylated camphorsultam represented by the general formula [I].

The hydrolysis reaction of the present invention can be preferably carried out by mixing the acylated camphorsultam represented by the general formula [I], the branched alkanol, water, and the alkaline earth metal hydroxide, and then heating the mixture or preparing a branched-alkanol solution of the acylated camphorsultam represented by the general formula [I] and an aqueous solution of alkaline earth metal hydroxide previously, mixing the two solutions, and then heating the mixture. The heating temperature (reaction temperature) is preferably within the range from 40° C. to the reflux temperature of the solvent, and more preferably within the range from 50° C. to the reflux temperature of the solvent.

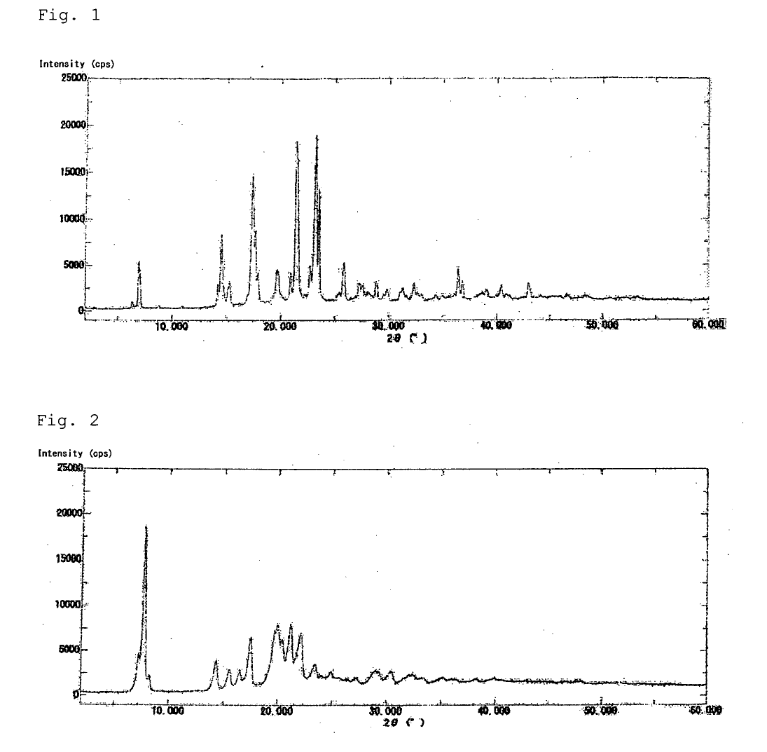

Thus, the optically active carboxylic acid compound represented by the general formula [II], which is a hydrolysate in the reaction solution. The optically active carboxylic acid compound represented by the general formula [II] can be converted into a suitable salt (for example, an organic amine salt such as a cyclohexylamine salt) as needed, and then can be isolated as the optically active carboxylic acid compound represented by the general formula [II] or the salt thereof by, for example, cooling, crystallization and filtration. For example, by obtaining crude optically active carboxylic acid compound represented by the general formula [II] from the reaction solution after hydrolysis, then adding dropwise a heptane solution thereof into cyclohexylamine or a solution thereof at about 20° C., then cooling the mixture, and then collecting precipitated crystal by filtration, the optically active carboxylic acid compound represented by the general formula [II] with high purity can be isolated as a cyclohexylamine salt (XRD charts thereof are shown in FIGS. 1 and 2). From the resultant salt, according to a conventional method, the free optically active carboxylic acid compound represented by the general formula [II] can be obtained.

Also, in the reaction solution, camphorsultam is produced as a by-product. This camphorsultam can be isolated from the post-reaction solution and be purified according to a conventional method. The isolated and purified camphorsultam can be reused to produce the acylated camphorsultam represented by the general formula [I].

In the case the optically active carboxylic acid compound represented by the general formula [II] obtained by the process of the present invention is, for example, (2S)-2-(2-propenyl) octanoic acid or the salt thereof including cyclohexylamine salts, reduction of the compound according to the method disclosed in WO99/58513 and WO00/48982 can give (2R)-2-propyl octanoic acid or a salt thereof, which is useful as a medicament.

### EXAMPLES

The present invention will, hereinafter, be described in detail by way of Examples, but it is to be construed that the present invention is in no way limited to these Examples. The raw compound, N-[(2S)-2-(2-propenyl)octanoyl]-(1S)-(−)-2,10-camphorsultam, which is used in the below Examples and Comparative Example, is represented by the following structural formula,

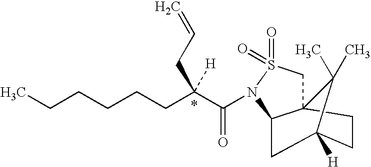

and according to the IUPAC nomenclature system, it is (3aS,6R,7aR)-1-[(2S)-2-allyloctanoyl]-8,8-dimethylhexahydro-3a,6-methano-2,1-benzoisothiazole 2,2-dioxide. Also, (1S)-(−)-2,10-camphorsultam, which is produced as a by-product, is (3aS,6R,7aR)-8,8-dimethylhexahydro-3a,6-methano-2,1-benzois othiazole 2,2-dioxide, according to the IUPAC nomenclature system. Further, (1S)-(−)-2,10-camphorsulfonimine (imine form), which coexists with (1S)-(−)-2,10-camphorsultam in the Comparative Example, is represented by the following structural formula,

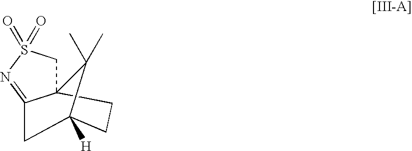

and according to the IUPAC nomenclature system, it is (3aS,6R)-8,8-dimethyl-4,5,6,7-tetrahydro-3a,6-methano-2,1-b enzoisothiazole 2,2-dioxide.

### Example 1

### (1) Preparation of (2S)-2-(2-propenyl) Octanoic Acid Cyclohexylamine Salt

### Calcium Hydroxide Method

In a round-bottomed flask, 40.00 g (0.104 mol) of (3aS,6R,7aR)-1-[(2S)-2-allyloctanoyl]-8,8-dimethylhexahydro-3a,6-methano-2,1-benzoisothiazole 2,2-dioxide, 200 ml of isopropanol, 76 ml of water and 15.53 g (0.210 mol) of calcium hydroxide were placed. The mixture was heated to the reflux temperature (about 82° C.), and was maintained at the same temperature for 16 hours. The reaction mixture was added dropwise into a liquid mixture of 80 ml of water and 54.60 g (0.524 mol) of 35% hydrochloric acid at 10 to 24° C., and then 200 ml of methyl tert-butyl ether was inpoured thereto. After the mixture was stirred for 10 min, the water layer was removed by separation, and then the organic layer was washed twice with 200 ml of water. After the organic layer of which amount was corresponding to 0.103 mol was vacuum-concentrated, an operation cycle of adding 135 ml of n-heptane to the residue and vacuum concentration was repeated three times. Next, 135 ml of n-heptane was added to the concentrate, and then the crystal was filtered. The obtained crystal was washed with 39.7 ml of n-heptane, and then dried under reduced pressure. Thus, 21.34 g of (1S)-(−)-2,10-camphorsultam (95.0% based on (3aS,6R,7aR)-1-[(2S)-2-allyloctanoyl]-8,8-dimethylhexahydro-3a,6-methano-2,1-benzoisothiazole 2,2-dioxide; GC area percentage value: 99.47%) was recovered.

Meanwhile, the filtrate was concentrated, and n-heptane was added thereto, to obtain 71.69 g (corresponding to 0.103 mol) of (2S)-2-(2-propenyl) octanoic acid heptane solution. From this of which amount was corresponding to 0.099 mol, cyclohexylamine salt of (2S)-2-(2-propenyl) octanoic acid was derived.

To be more specific, 273 ml of ethyl acetate and 8.81 g (0.089 mol) of cyclohexylamine were placed in a round-bottomed flask, and 10.19 g (corresponding to 0.015 mol) of the above-obtained (2S)-2-(2-propenyl) octanoic acid/heptane solution was added dropwise thereto at 20° C. over a 30 minute period, followed by stirring at 20 to 21° C. for 1 hour. Further, 57.76 g (corresponding to 0.084 mol) of (2S)-2-(2-propenyl) octanoic acid/heptane solution was added dropwise thereto at 20 to 21° C. over 2 hours and 30 min. Then, the resultant mixture was cooled and kept within the range of −3 to 3° C. for 1 hour. The precipitated crystal was collected by filtration, washed with 73 ml of cooled ethyl acetate, and dried under reduced pressure. Thus, 23.03 g of cyclohexylamine salt of (2S)-2-(2-propenyl) octanoic acid (yield based on (3aS,6R,7aR)-1-[(2S)-2-allyloctanoyl]-8,8-dimethylhexahydro-3a,6-methano-2,1-benzoisothiazole 2,2-dioxide: 82.3%; optical purity: 100%) was obtained.

### (2) Purification of Recovered Camphorsultam

In a round-bottomed flask, 10.00 g (0.046 mol) of recovered (1S)-(−)-2,10-camphorsultam, 10 ml of isopropanol, and 0.30 g of activated carbon were placed. The mixture was heated to the reflux temperature (81 to 82° C.), kept at the same temperature for 30 min, and filtered to remove the activated carbon, and the active carbon was washed with 5 ml of heated isopropanol. After 20.0 ml of water was added dropwise to the filtrate at 81 to 83° C. over 1 hour, the solution was cooled to 50° C., and after crystallization was seen, stirring was continued at 49 to 50° C. for 30 min. Then, the mixture was cooled and kept within the range of 2 to 5° C. for 1 hour. The precipitated crystal was collected by filtration, washed with cooled isopropanol-water (3.8 ml of isopropanol, and 5 ml of water), and dried under reduced pressure. Thus, 7.61 g of (1S)-(−)-2,10-camphorsultam (yield based on (3aS,6R,7aR)-1-[(2S)-2-allyloctanoyl]-8,8-dimethylhexahydro-3a,6-methano-2,1-benzoisothiazole 2,2-dioxide: 72.3%; GC area percentage value: 100.0%; content: 101.4%) was obtained.

In this Example, the following Example 2 and Comparative Example, Shimadzu GC-17A (Column: C4A-802 manufactured by GL Science, Detector: FID) was employed for gas chromatography.

### Example 2

### (1) Preparation of Cyclohexylamine Salt of (2S)-2-(2-propenyl) octanoic acid

### Barium Hydroxide Method

In a round-bottomed flask, 40.00 g (0.104 mol) of (3aS,6R,7aR)-1-[(2S)-2-allyloctanoyl]-8,8-dimethylhexahydro-3a,6-methano-2,1-benzoisothiazole 2,2-dioxide, 200 ml of isopropanol, 45.8 ml of water and 66.14 g (0.210 mol) of barium hydroxide octahydrate were placed. The mixture was heated to 50° C., and was maintained at the same temperature for 3 hours. The reaction mixture was added dropwise into a liquid mixture of 160 ml of water and 54.60 g (0.524 mol) of 35% hydrochloric acid at 10 to 24° C., and then 200 ml of methyl tert-butyl ether was inpoured thereto. After the mixture was stirred for 10 min, the water layer was removed by separation, and then the organic layer was washed once with 200 ml of water and once with 202.0 g of saline solution (200 ml of water, 2.0 g of NaCl). After the organic layer of which amount was corresponding to 0.103 mol was vacuum-concentrated, 135 ml of n-heptane was added to the residue, and vacuum concentration was carried out again. Next, 135 ml of n-heptane was added to the concentrate, and then the crystal was filtered. The obtained crystal was washed with 39.7 ml of n-heptane, and then dried under reduced pressure. Thus, 19.94 g of (3aS,6R,7aR)-8,8-dimethylhexahydro-3a,6-methano-2,1-benzois othiazole 2,2-dioxide, i.e. (1S)-(−)-2,10-camphorsultam was recovered (88.9% to (3aS,6R,7aR)-1-[(2S)-2-allyloctanoyl]-8,8-dimethylhexahydro-3a,6-methano-2,1-benzoisothiazole 2,2-dioxide; GC area percentage: 99.14%). Meanwhile, the filtrate was concentrated, and n-heptane was added thereto, to obtain 71.69 g (corresponding to 0.103 mol) of (2S)-2-(2-propenyl) octanoic acid/heptane solution. From this of which amount was corresponding to 0.099 mol, cyclohexylamine salt of (2S)-2-(2-propenyl) octanoic acid was derived.

Separately, 273 ml of ethyl acetate and 8.81 g (0.089 mol) of cyclohexylamine were placed in a round-bottomed flask, and 10.19 g (corresponding to 0.015 mol) of the (2S)-2-(2-propenyl) octanoic acid/heptane solution was added dropwise thereto at 20° C. over a 30 minute period, followed by stirring at 20 to 21° C. for 1 hour. Further, 57.76 g (corresponding to 0.084 mol) of (2S)-2-(2-propenyl) octanoic acid/heptane solution was added dropwise thereto at 20 to 21° C. over 2 hours and 30 min. Then, the resultant mixture was cooled and kept within the range of −3 to 3° C. for 1 hour. The crystal was collected by filtration, washed with 73 ml of cooled ethyl acetate, and dried under reduced pressure. Thus, 22.67 g of cyclohexylamine salt of (2S)-2-(2-propenyl) octanoic acid (yield based on (3aS,6R,7aR)-1-[(2S)-2-allyloctanoyl]-8,8-dimethylhexahydro-3a,6-methano-2,1-benzoisothiazole 2,2-dioxide: 81.0%; optical purity: 100%) was obtained.

### (2) Purification of Recovered Camphorsultam

In a round-bottomed flask, 10.00 g (0.046 mol) of recovered (1S)-(−)-2,10-camphorsultam, 10 ml of isopropanol, and 0.30 g of activated carbon were placed. The mixture was heated to the reflux temperature (81 to 82° C.), kept for 30 min, and filtered to remove the activated carbon, and the active carbon was washed with 5 ml of heated isopropanol. After 20.0 ml of water was added dropwise to the filtrate at 81 to 82° C. over 1 hour, the solution was cooled to 50° C., and after crystallization was seen, stirring was continued at 49 to 50° C. for 30 min. Then, the mixture was cooled and kept within the range of 2 to 5° C. for 1 hour. The crystal was collected by filtration, washed with cooled isopropanol-water (3.8 ml of isopropanol, 5 ml of water), and dried under reduced pressure. Thus, 7.39 g of (1S)-(−)-2,10-camphorsultam (yield based on (3aS,6R,7aR)-1-[(2S)-2-allyloctanoyl]-8,8-dimethylhexahydro-3a,6-methano-2,1-benzoisothiazole 2,2-dioxide: 65.7%; GC area percentage value: 100.0%; content: 101.4%) was obtained.

### Example 3 to 6

According to the same manner as in Example 1 or 2, hydrolysis was performed using 100 parts of (3aS,6R,7aR)-1-[(2S)-2-allyloctanoyl]-8,8-dimethylhexahydro-3a,6-methano-2,1-benzoisothiazole 2,2-dioxide, under the conditions shown in the following Table 1. The amount of (2S)-2-(2-propenyl) octanoic acid produced in the reaction solution was measured by LC, and the production rate thereof was calculated. The results are shown in the following Table 1.

TABLE 1

| | Solvent | | Water | Base | | | | |
|---|---|---|---|---|---|---|---|---|
| Example | | Part by | Part by | | Part by | Reaction | Reaction | Production |
| No. | Type | volume | volume | Type | mol | Temperature | Time | Rate (%) |
| 3 | IPA | 500 | 194 | Ba(OH)₂ | 2.0 | 82° C. | 1 h | 86.1 |
| | | | | | | (Reflux) | | |
| 4 | IPA | 500 | 194 | Ba(OH)₂ | 2.0 | 60° C. | 1 h | 87.5 |
| 5 | t-BuOH | 500 | 194 | Ba(OH)₂ | 2.0 | 50° C. | 4 h | 82.3 |
| 6 | IPA | 500 | 194 | Sr(OH)₂ | 2.0 | 82° C. | 4 h | 88.0 |
| | | | | | | (Reflux) | | |

The IPA and t-BuOH in the table represent isopropanol and tertiary butanol respectively.

(2S)-2-(2-propenyl) octanoic acid produced in Examples 3 to 6 was derived to cyclohexylamine salt. The optical purity measured was 100% in each Example.

### Comparative Example 1

### Preparation of Cyclohexylamine Salt of (2S)-2-(2-propenyl) Octanoic Acid

### Hydrogen Peroxide/Potassium Hydroxide Method

In a round-bottomed flask, 40.00 g (0.105 mol) of (3aS,6R,7aR)-1-[(2S)-2-allyloctanoyl]-8,8-dimethylhexahydro-3a,6-methano-2,1-benzoisothiazole 2,2-dioxide and 160 ml of diglyme were placed, and the mixture was cooled to 17° C. while nitrogen was being sent thereto with the intention of limiting the concentration of oxygen to 5% or less until the end of the reaction. After 0.51 g (0.005 mol) of 35% hydrogen peroxide solution was inpoured, 15.79 g (0.162 mol) of 35% aqueous hydrogen peroxide solution and 19.61 g (0.168 mol) of 48% aqueous potassium hydroxide solution were simultaneously inpoured at 17° C. over 9 hours, followed by stirring at 17° C. for 2 hours, and the end of the reaction was checked by HPLC.

Separately, 21.14 g (0.168 mol) of sodium sulfite was dissolved in 110 ml of water in a flask with a stop-cock at room temperature, 58.97 g (0.566 mol) of 35% hydrochloric acid was added dropwise at 9 to 10° C., and then the above-mentioned reaction mixture was added dropwise thereto at 5 to 12° C. over 1 hour. After the resultant mixture was heated to 21° C. and stirred at the same temperature for 35 min, 200 ml of methyl tert-butyl ether was inpoured thereto, the mixture was stirred for 10 min, and the water layer was removed by separation. The organic layer was washed with saline solution (1.60 g NaCl, 200 ml tap water), 2.40 g of n-heptane and saline solution (1.60 g NaCl, 200 ml tap water) were added thereto, the mixture was stirred for 15 min, the water layer was removed by separation, and the end of washing was checked by GC. The organic layer was concentrated at normal pressures, and 240 ml of n-heptane was added to the obtained residue, which was then vacuum-concentrated until the residue was reduced to about 160 ml. Then, the residue was cooled and stirred at 0 to 10° C. for 30 minutes. The obtained crystal was filtered, washed with 40 ml of n-heptane, and dried under reduced pressure. Thus, 6.48 g of camphorsultam (yield based on (3aS,6R,7aR)-1-[(2S)-2-allyloctanoyl]-8,8-dimethylhexahydro-3a,6-methano-2,1-benzoisothiazole 2,2-dioxide: 28.7%; GC area percentage value: 98.23%) was obtained. (1S)-2,10-camphorsulfonimine (imine form) was contained in the recovered camphorsultam and couldn't be removed by purification.

Meanwhile, the filtrate was concentrated under reduced pressure and n-heptane was added thereto, to obtain 72.17 g (corresponding to 0.105 mol) of (2S)-2-(2-propenyl) octanoic acid/heptane solution. From this of which amount was corresponding to 0.087 mol, cyclohexylamine salt of (2S)-2-(2-propenyl) octanoic acid was derived.

In a round-bottomed flask, 241 ml of ethyl acetate, 7.78 g (0.078 mol) of cyclohexylamine were placed and 9.00 g (0.013 mol) of (2S)-2-(2-propenyl) octanoic acid/heptane solution was added dropwise thereto at 20° C. over a 30 minute period, followed by stirring at 20 to 21° C. for 1 hour. Further, 51.00 g (corresponding to 0.074 mol) of (2S)-2-(2-propenyl) octanoic acid/heptane solution was added dropwise thereto at 20° C. over 2 hour 30 min. Then, the mixture was cooled and kept within the range of −3 to 3° C. for 1 hour. The crystal was collected by filtration, washed with 64 ml of cooled ethyl acetate, and dried under reduced pressure. Thus, 20.29 g of cyclohexylamine salt of (2S)-2-(2-propenyl) octanoic acid (yield based on (3aS,6R,7aR)-1-[(2S)-2-allyloctanoyl]-8,8-dimethylhexahydro-3a,6-methano-2,1-benzoisothiazole 2,2-dioxide: 82.1%; optical purity: 100%) was obtained.

### Example of Raw Material Preparation

### (3aS,6R,7aR)-1-[(2S)-2-allyloctanoyl]-8,8-dimethylhexahydro-3a,6-methano-2,1-benzoisothiazole 2,2-dioxide

### (1)(3aS,6R,7aR)-8,8-dimethyl-1-octanoylhexahydro-3a,6-methano-2,1-benzoisothiazole 2,2-dioxide

In a round-bottomed flask, 80 g of (1S)-(−)-2,10-camphorsultam, 236.4 g of methyl tert-butyl ether, 2.27 g of 4-(dimethylamino)-pyridine and 41.36 g of triethylamine were placed, and to this, 63.46 g of capryloyl chloride was added dropwise at room temperature. After stirring for 1 hour, the reaction solution was added to a mixed solution of 7.75 g of 35% hydrochloric acid and 200 g of water, and the organic layer was separated. The organic layer was washed with 25% aqueous sodium hydroxide solution and subsequently with water, and concentrated under reduced pressure. Thus, 125.34 g of the titled compound was obtained (quality: 99.7% measured by GC).

Gas chromatography analysis was carried out using Shimadzu GC-14A (Column: 5% Silicone OV-17 manufactured by GL Science, Detector: FID).

### (2)(3aS,6R,7aR)-1-[(2S)-2-allyloctanoyl]-8,8-dimethylhexahydro-3a,6-methano-2,1-benzoisothiazole 2,2-dioxide

To the compound obtained in the above (1), 92.75 g of methyl tert-butyl ether was added to make the total weight 218.0 g. With nitrogen being supplied, 213.92 g of the (3aS,6R,7aR)-8,8-dimethyl-1-octanoylhexahydro-3a,6-methano-2,1-benzoisothiazole 2,2-dioxide/methyl tert-butyl ether solution obtained in the above, 61.64 g of 1,3-dimethylimidazolidine and 226.24 g of tetrahydrofuran were placed into a round-bottom flask, and the mixture was cooled to −72° C. To this, 143.94 g of 17.15% n-BuLi/n-hexane solution was added dropwise at −72 to −66° C., and the mixture was stirred at the same temperature for 30 min. To this, 65.33 g of allyl bromide was added dropwise at −74 to −69° C., the mixture was heated up to −20° C. and allowed to react at the same temperature for 15 hours. The end of the reaction was checked by LC. The mixture was heated up to −5° C. and stirred at −5 to 1° C. for 2 hours (reaction solution A).

In a round-bottomed flask, 241 g of water and 45.00 g (0.431 mol) of 35% hydrochloric acid were placed, to this, the above reaction solution A was added dropwise at 19 to 21° C., the flask was washed with 22.93 g of tetrahydrofuran and wash liquid was added thereto, and then layers were separated. The organic layer was washed with saline solution obtained by dissolving 2.58 g of sodium chloride in 258.2 g of water, followed by separation. After the organic layer was concentrated under reduced pressure and 764.59 g of isopropanol was added to the residue, vacuum concentration was carried out again. 522.02 g of isopropanol was added to the residue, heated to 65° C. to dissolve the residue, and then, to this, 681 g of water was added dropwise. The solution was cooled and stirred at 57° C. for 2 hours, followed by gradual cooling to 10° C. and stirring at 5 to 10° C. for 1 hour, and then crystal was filtered. The obtained crystal was washed with a mixed solution of 86.82 g of isopropanol and 61.5 g of water and dried under reduced pressure. 675.88 g of isopropanol was added to 105.00 g of the crystal, and the mixture was heated to dissolve the crystal, and then, to this, 315 g of water was added dropwise. Then, the solution was cooled and stirred at 57° C. for 2 hours, followed by gradual cooling to 10° C. and stirring at 5 to 10° C. for 1 hour. The precipitated crystal was filtered, washed with a mixed solution of 65.94 g of isopropanol and 31.5 g of water, and dried under reduced pressure. Thus, 99.7 g of the titled compound (yield based on camphorsultam: 74.7%; quality: 99.99% d.e (measured by HPLC)) was obtained.

HPLC analysis was carried out using Shimadzu LC-10A (Column: CHIRALCELL OJ-RH manufactured by Daicel chemical industries, LTD.; mobile phase: acetonitrile solution; wavelength: 210 nm).

### INDUSTRIAL APPLICABILITY

According to the present invention, from an acylated camphorsultam, a corresponding optically active carboxylic acid compound can be produced safely and at low cost. The camphorsultam obtained as a by-product can be recovered and reused in the production of raw materials.

1. A process for producing an optically active carboxylic acid compound represented by the general formula [II],
R—COOH [II]
wherein, R represents an optically active hydrocarbon group having at least one asymmetric carbon atom and optionally having at least one substituent, which comprises hydrolyzing an acylated camphorsultam represented by the general formula [I] 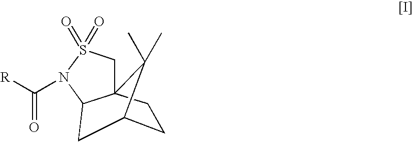
with an aqueous solution of an alkaline earth metal hydroxide in the presence of a branched alkanol, wherein, R has the same meaning as described above.

2. The process according to claim 1, wherein the alkaline earth metal hydroxide is barium hydroxide, calcium hydroxide or strontium hydroxide.

3. The process according to claim 1, wherein the branched alkanol is a secondary alkanol or a tertiary alkanol.

4. The process according to claim 1, wherein the branched alkanol is isopropanol or tertiary butanol.

5. The process according to claim 1, wherein the alkaline earth metal hydroxide is barium hydroxide or calcium hydroxide, and the branched alkanol is isopropanol.

6. The process according to claim 1, wherein R is an optically active hydrocarbon group having at least one asymmetric carbon atom and optionally having at least one substituent selected from the group consisting of an alkyl group, an alkoxy group, an optionally protected hydroxy group, an aryl group, an aryloxy group, an aralkyloxy group, an optionally protected amino group, a monoalkylamino group, a dialkylamino group, an acylamino group, an oxo group, an alkylthio group, and an arylthio group.

7. The process according to claim 1, wherein the hydrocarbon group is a saturated or unsaturated acyclic hydrocarbon group, or a saturated or unsaturated cyclic hydrocarbon group.

8. The process according to claim 7, wherein the saturated or unsaturated acyclic hydrocarbon group is a linear or branched alkyl group, a linear or branched alkenyl group, or a linear or branched alkynyl group.

9. The process according to claim 1, wherein R is an optically active branched alkenyl group having at least one asymmetric carbon atom and optionally having at least one substituent.

10. The process according to claim 1, wherein R is a group represented by the formula, 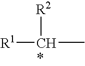
wherein R¹ represents a C1 to C10 alkyl group; R² represents a C1 to C6 alkyl group, a C2 to C6 alkenyl group, or a C2 to C6 alkynyl group; and * represents an asymmetric carbon atom.

11. The process according to claim 10, wherein R² is a C2 to C6 alkenyl group.

12. The process according to claim 1, wherein the acylated camphorsultam represented by the general formula [I] is an acylated camphorsultam represented by the formula [1-A] 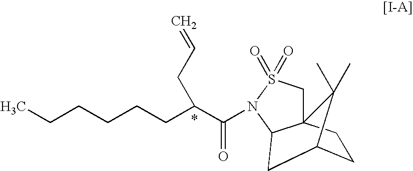
wherein * represents an asymmetric carbon atom,
and the optically active carboxylic acid compound represented by the general formula [II] is an optically active 2-(2-propenyl)octanoic acid represented by the following formula [II-A] 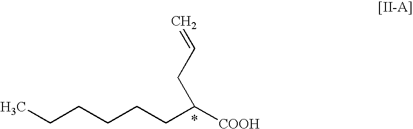
wherein * represents an asymmetric carbon atom.

13. The process according to claim 12, wherein the configurations of asymmetric carbon atoms bound to the 2-propenyl group in the optically active 2-(2-propenyl)octanoic acid represented by the formula [II-A] and in the acylated camphorsultam represented by the formula [1-A] are S configurations.

14. The process according to claim 12, wherein the branched alkanol is isopropanol and the alkaline earth metal hydroxide is calcium hydroxide.

15. The process according to claim 1, wherein the camphorsultam represented by the formula [III] 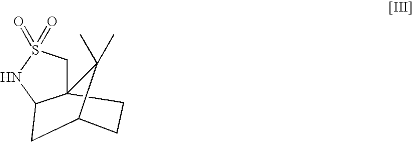
is additionally recovered from the post-reaction solution after hydrolysis.

16. The process according to claim 13, wherein the branched alkanol is isopropanol and the alkaline earth metal hydroxide is calcium hydroxide.
